SEQUENCE LISTING (42 sequences)

SEQ ID NO: 1 - protein, 31 aa, Homo sapien
  SITE 1: NHR; wherein R is hydrogen or a linear or branched chain alkyl, acyl or aryl group
SVSEIQLMHNLGKHLNSMERVEWLRKKLQDV

SEQ ID NO: 2 - protein, 32 aa, Homo sapien
  SITE 1: NHR; wherein R is hydrogen or a linear or branched chain alkyl, acyl or aryl group
SVSEIQLMHNLGKHLNSMERVEWLRKKLQDVH

SEQ ID NO: 3 - protein, 33 aa, Homo sapien
  SITE 1: NHR; wherein R is hydrogen or a linear or branched chain alkyl, acyl or aryl group
SVSEIQLMHNLGKHLNSMERVEWLRKKLQDVHN

SEQ ID NO: 4 - protein, 34 aa, Homo sapien
  SITE 1: NHR; wherein R is hydrogen or a linear or branched chain alkyl acyl or aryl group
SVSEIQLMHNLGKHLNSMERVEWLRKKLQDVHNF

SEQ ID NO: 5 - protein, 35 aa, Homo sapien
  SITE 1: NHR; wherein R is hydrogen or a linear or branched chain alkyl, acyl or aryl group
SVSEIQLMHNLGKHLNSMERVEWLRKKLQDVHNFV

SEQ ID NO: 6 - protein, 36 aa, Homo sapien
  SITE 1: NHR; wherein R is hydrogen or a linear or branched chain alkyl, acyl or aryl group
SVSEIQLMHNLGKHLNSMERVEWLRKKLQDVHNFVA

SEQ ID NO: 7 - protein, 37 aa, Homo sapien
  SITE 1: NHR; wherein R is hydrogen or a linear or branched chain alkyl, acyl or aryl group
SVSEIQLMHNLGKHLNSMERVEWLRKKLQDVHNFVAL

SEQ ID NO: 8 - protein, 38 aa, Homo sapien
  SITE 1: NHR; wherein R is hydrogen or a linear or branched chain alkyl, acyl or aryl group
SVSEIQLMHNLGKHLNSMERVEWLRKKLQDVHNFVALG

SEQ ID NO: 9 - protein, 39 aa, Homo sapien
  SITE 1: NHR; wherein R is hydrogen or a linear or branched chain alkyl, acyl or aryl group
SVSEIQLMHNLGKHLNSMERVEWLRKKLQDVHNFVALGA

SEQ ID NO: 10 - protein, 40 aa, Homo sapien
  SITE 1: NHR; wherein R is hydrogen or a linear or branched chain alkyl, acyl or aryl group
SVSEIQLMHNLGKHLNSMERVEWLRKKLQDVHNFVALGAP

SEQ ID NO: 11 - protein, 41 aa, Homo sapien
  SITE 1: NHR; R is hydrogen or a linear or branched chain alkyl, acyl or aryl group
SVSEIQLMHNLGKHLNSMERVEWLRKKLQDVHNFVALGAPL

SEQ ID NO: 12 - protein, 42 aa, Homo sapien
  SITE 1: NHR; wherein, R is hydrogen or a linear or branched chain alkyl, acyl or aryl group
SVSEIQLMHNLGKHLNSMERVEWLRKKLQDVHNFVALGAPLA

SEQ ID NO: 13 - protein, 43 aa, Homo sapien
  SITE 1: NHR; R is hydrogen or a linear or branched chain alkyl, acyl or aryl group
SVSEIQLMHNLGKHLNSMERVEWLRKKLQDVHNFVALGAPLAP

SEQ ID NO: 14 - protein, 44 aa, Homo sapien
  SITE 1: NHR; R is hydrogen or a linear or branched chain alkyl, acyl or aryl group
SVSEIQLMHNLGKHLNSMERVEWLRKKLQDVHNFVALGAPLAPR

SEQ ID NO: 15 - protein, 31 aa, Homo sapien
  SITE (22)...(26): cyclo Glu22-Lys26
SVSEIQLMHNLGKHLNSMERVEWLRKLLQDV

SEQ ID NO: 16 - protein, 31 aa, Homo sapien
  SITE (13)...(17): cyclo Lys13-Glu17
SVSEIQLMHNLGKHLNEMERVEWLRKLLQDV

SEQ ID NO: 17 - protein, 84 aa, Homo sapien
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 18 - protein, 31 aa, Homo sapien
  SITE 1: NHR; wherein R is hydrogen or a linear or branched chain alky, acyl or aryl
XVSEIQLXHNLGXXXXXXERVXWLXXXLQDV

SEQ ID NO: 19 - protein, 31 aa, Homo sapien
  SITE 1: NHR; where R is hydrogen or a linear or branched chain alkyl acyl or aryl group
XVSEIQLMHNLGKHLNSMERVEWLRKKLQDV

SEQ ID NO: 20 - protein, 31 aa, Homo sapien
  SITE 1: NHR; wherein R is hydrogen or a linear or branched chain alkyl, acyl or aryl group
SVSEIQLXHNLGKHLNSMERVEWLRKKLQDV

SEQ ID NO: 21 - protein, 31 aa, Homo sapien
  SITE 1: NHR; where R is hydrogen or a linear or branched chain alkyl, acyl or aryl group
SVSEIQLMHNLGXHLNSMERVEWLRKKLQDV

SEQ ID NO: 22 - protein, 31 aa, Homo sapien
  SITE 1: NHR; where R is hydrogen or a linear branched chain alkyl, acyl or aryl group
SVSEIQLMHNLGKXLNSMERVEWLRKKLQDV

SEQ ID NO: 23 - protein, 31 aa, Homo sapien
  SITE 1: NHR; where R is hydrogen or a linear or branched chain alkyl, acyl or aryl group
SVSEIQLMHNLGKHXNSMERVEWLRKKLQDV

SEQ ID NO: 24 - protein, 31 aa, Homo sapien
  SITE 1: NHR; where R is hydrogen or a linear or branched chain alkyl, acyl or aryl
SVSEIQLMHNLGKHLXSMERVEWLRKKLQDV

SEQ ID NO: 25 - protein, 31 aa, Homo sapien
  SITE 1: NHR; where R is hydrogen or a linear or branched chain alkyl, acyl or aryl group
SVSEIQLMHNLGKHLNXMERVEWLRKKLQDV

SEQ ID NO: 26 - protein, 31 aa, Homo sapien
  SITE 1: NHR; wherein R is hydrogen or a linear or branched chain alkyl, acyl or aryl group
SVSEIQLMHNLGKHLNSXERVEWLRKKLQDV

SEQ ID NO: 27 - protein, 31 aa, Homo sapien
  SITE 1: NHR, where R is hydrogen or a linear or branched chain alkyl, acyl or aryl group
SVSEIQLMHNLGKHLNSMERVXWLRKKLQDV

SEQ ID NO: 28 - protein, 31 aa, Homo sapien
  SITE 1: NHR; wherein R is hydrogen or a linear or branched chain alkyl, acyl or aryl group
SVSEIQLMHNLGKHLNSMERVEWLXKKLQDV

SEQ ID NO: 29 - protein, 31 aa, Homo sapien
  SITE 1: NHR; wherein R is hydrogen or a linear or branched chain alkyl, acyl or aryl group
SVSEIQLMHNLGKHLNSMERVEWLRXKLQDV

SEQ ID NO: 30 - protein, 31 aa, Homo sapien
  SITE 1: NHR; where R is hydrogen or a linear or branched chain alkyl, acyl or aryl group
SVSEIQLMHNLGKHLNSMERVEWLRKXLQDV

SEQ ID NO: 31 - protein, 31 aa, Homo sapien
  SITE 1: NHR; where R is hydrogen or a linear or branched chain alkyl, acyl or aryl group
SVSEIQLMHNLGKHLNSMERVXWLRXKLQDV

SEQ ID NO: 32 - protein, 31 aa, Homo sapien
  SITE 1: NHR; where R is hydrogen or a linear or branched chain alkyl, acyl or aryl group
SVSEIQLMHNLGKHLNSMERVXWLRXXLQDV

SEQ ID NO: 33 - protein, 31 aa, Homo sapien
  SITE 1: NHR; where R is hydrogen or a linear or branched chain alkyl, acyl or aryl
SVSEIQLMHNLGXHLNXMERVXWLRXXLQDV

SEQ ID NO: 34 - protein, 31 aa, Homo sapien
  SITE 1: NHR; where R is hydrogen or a linear or branched chain alkyl, acyl or aryl group
SVSEIQLXHNLGKHLNSXERVXWLXXXLQDV

SEQ ID NO: 35 - protein, 31 aa, Homo sapien
  SITE 1: NHR; where R is hydrogen or a linear or branched chain alkyl, acyl or aryl group
SVSEIQLXHNLGXHLNXXERVXWLXXXLQDV

SEQ ID NO: 36 - protein, 31 aa, Homo sapien
  SITE 1: NHR; where R is hydrogen or a linear or branched chain alkyl, acyl or aryl group
SVSEIQLMHNLGKHLNEMERVEWLRKXLQDV

SEQ ID NO: 37 - protein, 32 aa, Homo sapien
  SITE (22)...(26): cycle Glu22-Lys26
SVSEIQLMHNLGKHLNSMERVEWLRKLLQDVH

SEQ ID NO: 38 - protein, 33 aa, Homo sapien
  SITE (22)...(26): cyclo Glu22-Lys26
SVSEIQLMHNLGKHLNSMERVEWLRKLLQDVHN

SEQ ID NO: 39 - protein, 34 aa, Homo sapien
  SITE (22)...(26): cyclo Glu22-Lys26
SVSEIQLMHNLGKHLNSMERVEWLRKLLQDVHNF

SEQ ID NO: 40 - protein, 32 aa, Homo sapien
  SITE (13)...(17): cyclo Lys13-Glu17
SVSEIQLMHNLGKHLNEMERVEWLRKLLQDVH

SEQ ID NO: 41 - protein, 33 aa, Homo sapien
  SITE (13)...(17): cyclo Lys13-Glu17
SVSEIQLMHNLGKHLNEMERVEWLRKLLQDVHN

SEQ ID NO: 42 - protein, 34 aa, Homo sapien
  SITE (13)...(17): cyclo Lys13-Glu17
SVSEIQLMHNLGKHLNEMERVEWLRKLLQDVHNF

(19) United States
(12) Patent Application Publication
(10) Pub. No.: US 2005/0065071 A1
(43) Pub. Date: Mar. 24, 2005

(54) Cyclic analogs of human parathyroid hormone for the treatment of conditions characterized by hyperproliferative skin cells

(75) Inventors: Paul Morley, Ottawa (CA); James F. Whitfield, Ottawa (CA)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: National Research Council of Canada, Ontario (CA)

(21) Appl. No.: 10/892,025

(22) Filed: Jul. 15, 2004

Related U.S. Application Data
(60) Provisional application No. 60/487,513, filed on Jul. 15, 2003.

(51) Int. Cl. A61K 38/29

(52) U.S. Cl. 514/9

(57) ABSTRACT

The present invention provides methods for treating conditions characterized by hyperproliferation of skin cells, by administering to an individual in need thereof a cyclic analog of human parathyroid hormone.

### RELATED APPLICATIONS

The application claims the benefit of U.S. Provisional Application No. 60/487,513, filed Jul. 15, 2003, the entire teachings of which are incorporated herein by reference.

### BACKGROUND OF THE INVENTION

Disorders characterized by hyperproliferation of skin cells are a group of conditions that affect many people throughout the world. Psoriasis, one example of such a disorder, is a disease of the epidermis and a major cause of disability and disfigurement for 1 to 3% of the population of the world. In the United States approximately 2,000,000 to 8,000,000 people suffer from psoriasis, and approximately 100,000 are severely affected.

Psoriasis is diagnosed by the presence of scaling, erythematous lesions on the scalp and extensor aspects of the arms and legs. Psoriatic lesions often are accentuated at sites of repeated trauma such as the elbows and knees. Furthermore, this skin disorder can afflict most of the areas of the skin of some individuals and can also cause internal damage such as arthritis. This disease is characterized by hyperproliferation of the basal cells (a several fold increase in the number of basal cells of the epidermis). This increase in the basal cell population reduces the turnover time of the epidermis from the normal 27 days to 3-4 days. This shortened interval prevents normal cell maturation and keratinization, and this failure of maturation is reflected in an array of abnormal morphologic and biochemical changes. Numerous cytologic, histologic, histochemical, and biochemical alterations are known to be the result, rather than the cause, of the disease process.

Other conditions characterized by hyperproliferation of skin cells include psoriatic arthritis, erythrokeratodermia variabilis, pityriasis rosea, lichen planus, and pityriasis rubra pilaris.

The prognosis of psoriasis and other conditions characterized by hyperproliferative skin depends on a number of factors, including the extent and severity of the onset of the condition. Generally, the condition is most severe when onset occurs at an early age. While acute occurrences of these skin conditions usually can be controlled, permanent remission is rare, and there is no cure for many of these conditions. In addition, not all therapies are effective on all patients in need of treatment of hyperproliferative skin disorders. Therefore, new therapeutic methods are needed for the treatment of conditions characterized by hyperproliferation of skin cells.

### SUMMARY OF THE INVENTION

The present invention provides new therapies for individuals in need of treatment for conditions characterized by hyperproliferative skin cells.

Accordingly, in one aspect, the invention features a method of treating a condition characterized by hyperproliferation of skin cells in an individual at risk for or having the condition comprising administering a therapeutically effective amount of a cyclic analog of human parathyroid hormone (hPTH) having the amino acid sequence: R-NH-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-Y (SEQ ID NO: 1), wherein R is hydrogen or a linear or branched chain alkyl, acyl, or aryl group; and Y is X, His-X, His-Asn-X, or His-Asn-Phe-X, wherein X is OR or NHR, and having 0, 1, 2, 3, 4, 5, 6, 7, 8, 9, 10, 11, or 12 amino acids in the analog differ from the amino acid in the corresponding position of SEQ ID NO:1 or a pharmaceutically acceptable salt thereof to the individual.

In another aspect, the invention features a method of treating a condition characterized by hyperproliferation of skin cells in an individual at risk for or having the condition comprising administering a therapeutically effective amount of the human parathyroid hormone hPTH analog cyclo(Glu²²-Lys²⁶)[Leu²⁷]-hPTH-(1-31)-NH₂ or a pharmaceutically acceptable salt thereof to the individual.

In another aspect, the invention features a method of treating a condition characterized by hyperproliferation of skin cells in an individual at risk for or having the condition comprising administering a therapeutically effective amount of a cyclic analog of human parathyroid hormone (hPTH) consisting of the amino acid sequence R-NH-Xaa1-Val-Ser-Glu-Ile-Gln-Leu-Xaa8-His-Asn-Leu-Gly-Xaa13-Xaa14-Xaa15-Xaa16-Xaa17-Xaa18-Glu-Arg-Val-Xaa22-Trp-Leu-Xaa25-Xaa26-Xaa27-Leu-Gln-Asp-Val-Y (SEQ ID NO: 18), wherein Xaa1 is selected from the group consisting of serine, alanine, and α-aminoisobutyric acid; Xaa8 is selected from the group consisting of methionine, norisoleucine, and a hydrophobic amino acid; Xaa13 is selected from the group consisting of lysine, ornithine, glutamic acid, aspartic acid, cysteine, and homocysteine; Xaa14 is histidine or a water soluble amino acid; Xaa15 is leucine or a water soluble amino acid; Xaa16 is asparagine or a water soluble amino acid; Xaa17 is selected from the group consisting of serine, glutamic acid, aspartic acid, lysine, ornithine, cysteine, homocysteine, and a water soluble amino acid; Xaa18 is selected from the group consisting of methionine, norisoleucine, and a hydrophobic amino acid; Xaa22 is selected from the group consisting of lysine, ornithine, glutamic acid, cysteine, aspartic acid, and homocysteine; Xaa25 is arginine or histidine; Xaa26 is selected from the group consisting of lysine, ornithine, glutamic acid, cysteine, aspartic acid, and homocysteine; and Xaa27 is selected from the group consisting of lysine, leucine, isoleucine, norisoleucine, alanine, methionine, and wherein R is hydrogen or a linear or branched chain alkyl, acyl, or aryl group; and Y is X, His-X, His-Asn-X, or His-Asn-Phe-X, wherein X is OR or NHR, or a pharmaceutically acceptable salt thereof to the individual.

In another aspect, the invention features a method of treating a condition characterized by hyperproliferation of skin cells in an individual at risk for or having the condition comprising administering a therapeutically effective amount of a cyclic analog of human parathyroid hormone (hPTH) of Formula I:
RNH-W-Z-B (I)
wherein R is hydrogen or a linear or branched chain alkyl, acyl, or aryl group; W is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val (SEQ ID NO: 1) having 0, 1, 2, 3, 4, 5, 6, 7, 8, 9, 10, 11 or 12 amino acids in the analog differ from the amino acid in the corresponding position of SEQ ID NO:1, Z is selected from the group consisting of His, His-Asn, His-Asn-Phe, His-Asn-Phe-Val (SEQ ID NO: 5), His-Asn-Phe-Val-Ala (SEQ ID NO: 6), His-Asn-Phe-Val-Ala-Leu (SEQ ID NO: 7), His-Asn-Phe-Val-Ala-Leu-Gly (SEQ ID NO: 8), His-Asn-Phe-Val-Ala-Leu-Gly-Ala (SEQ ID NO: 9), His-Asn-Phe-Val-Ala-Leu-Gly-Ala-Pro (SEQ ID NO: 10), His-Asn-Phe-Val-Ala-Leu-Gly-Ala-Pro-Leu (SEQ ID NO: 11), His-Asn-Phe-Val-Ala-Leu-Gly-Ala-Pro-Leu-Ala (SEQ ID NO: 12), His-Asn-Phe-Val-Ala-Leu-Gly-Ala-Pro-Leu-Ala-Pro (SEQ ID NO: 13), and His-Asn-Phe-Val-Ala-Leu-Gly-Ala-Pro-Leu-Ala-Pro-Arg (SEQ ID NO: 14), and B is OR or NHR, or a pharmaceutically acceptable salt thereof to the individual.

In another aspect, the invention features a method of inhibiting skin cell hyperproliferation by administering to the skin cell a cyclic analog of a parathyroid hormone as described herein.

### BRIEF DESCRIPTION OF THE DRAWINGS

FIG. 1 shows the structure of natural hPTH, residues 1-34 (hPTH-(1-34)) (SEQ ID NO: 4).

FIG. 2 shows the structure of natural hPTH-NH₂ residues 1-31 (SEQ ID NO: 1).

FIG. 3 shows the structure of [Leu²⁷]cyclo(Glu²²-Lys²⁶)-hPTH-(1-31)-NH₂ (SEQ ID NO: 15).

FIG. 4 shows the structure of [Glu¹⁷, Leu²⁷]cyclo(Lys¹³-Glu¹⁷, Glu²²-Lys²⁶)-hPTH-(1-31)-NH₂ (SEQ ID NO: 16).

FIG. 5 shows the amino acid sequence of human parathyroid hormone (hPTH) (SEQ ID NO: 17).

FIG. 6 is a histogram showing the effects of no treatment (Control) or of topical administration of 1 μg, 10 μg, or 50 μg of hPTH-(1-34) (1-34) or [Leu²⁷]cyclo(Glu²²-Lys²⁶)-hPTH-(1-31)-NH₂ (c1-c31) to SKH-1 mice on the proliferation of skin cells, as measured by ³H-thymidine incorporation (counts per minute/μg protein) in SKH-1 mice after 7 days of administration.

### DETAILED DESCRIPTION OF THE INVENTION

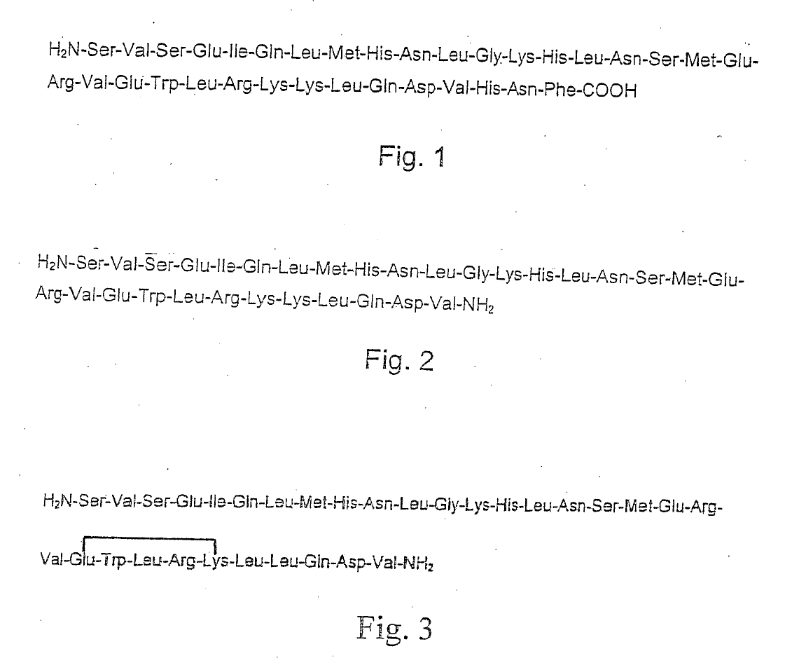

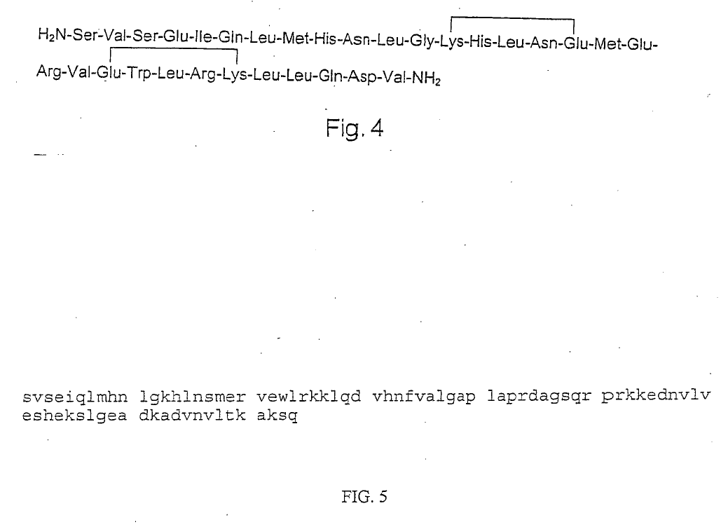

The present invention is based on the discovery that cyclic analogs of human parathyroid hormone inhibit skin cell proliferation. Therefore, cyclic analogs of hPTH can be used to treat conditions characterized by hyperproliferation of skin cells. As used herein a “cyclic analog of human parathyroid hormone” is a peptide having the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-Y, (SEQ ID NO: 1) wherein R is hydrogen or a linear or branched chain alkyl, acyl, or aryl group; and Y is X, His-X (hPTH-(1-32); SEQ ID NO: 2), His-Asn-X (hPTH-(1-33); SEQ ID NO: 3), or His-Asn-Phe-X (hPTH-(1-34); SEQ ID NO: 4), wherein X is OR or NHR, and wherein the peptide is cyclized by the coupling of one or more pairs of amino acids in the sequence. In one embodiment, the cyclic analog has the amino acid sequence of hPTH-(1-31) (FIG. 1; SEQ ID NO: 1). In preferred embodiments, R is H and/or Y is NH₂.

Skin is comprised of two layers, the dermis and the epidermis, and the cyclic analogs of the present invention can be used to inhibit cell proliferation of one or more types of cells that make up the skin, including basal cells located deep in the epidermis. When referring to the effect of any of the compositions or methods of the invention on the inhibition of proliferation of skin cells, the use of the terms “inhibit” or “decrease” encompasses at least a small but measurable reduction in skin cell proliferation. In preferred embodiments, skin cell proliferation is inhibited by at least 10%, 20%, 25%, 30%, 40%, 50%, 75%, 80%, or 90% over non-treated controls. Inhibition can be assessed using methods described herein, for example, ³H-thymidine incorporation, visual inspection of the area affected by the hyperproliferative skin disorder, histologic, cytologic, histochemical, or biochemical analysis or a sample taken from the affected area, or other methods known in the art. Such reductions in skin proliferation are capable of reducing the deleterious effects of a condition characterized by hyperproliferation of skin cells in in vivo embodiments.

Zero, 1, 2, 3, 4, 5, 6, 7, 8, 9, 10, 11, or 12 amino acids in the analog can differ from the amino acid in the corresponding position of SEQ ID NO:1. In one embodiment, the amino acid substitutions are limited to positions 13, 17, 22, 26, and/or 27. In another embodiment, 5 or fewer amino acids in the analog differ from the amino acid sequence of SEQ ID NO:1. In another embodiment, 0, 1, 2, or 3 amino acids in the analog differ from the amino acid sequence of SEQ ID NO:1.

In another embodiment, the analog consists of the amino acid sequence R-NH-Xaa1-Val-Ser-Glu-Ile-Gln-Leu-Xaa8-His-Asn-Leu-Gly-Xaa13-Xaa14-Xaa15-Xaa16-Xaa17-Xaa18-Glu-Arg-Val-Xaa22-Trp-Leu-Xaa25-Xaa26-Xaa27-Leu-Gln-Asp-Val-Y (SEQ ID NO: 18), wherein R and Y are as described above; Xaa1 is selected from the group consisting of serine, alanine, and α-aminoisobutyric acid; Xaa8 is selected from the group consisting of methionine, norisoleucine, and a hydrophobic amino acid; Xaa13 is selected from the group consisting of lysine, ornithine, glutamic acid, aspartic acid, cysteine, and homocysteine; Xaa14 is histidine or a water soluble amino acid; Xaa15 is leucine or a water soluble amino acid; Xaa16 is asparagine or a water soluble amino acid; Xaa17 is selected from the group consisting of serine, glutamic acid, aspartic acid, lysine, ornithine, cysteine, homocysteine, and a water soluble amino acid; Xaa18 is selected from the group consisting of methionine, norisoleucine, and a hydrophobic amino acid; Xaa22 is selected from the group consisting of lysine, ornithine, glutamic acid, cysteine, aspartic acid, and homocysteine; Xaa25 is arginine or histidine; Xaa26 is selected from the group consisting of lysine, ornithine, glutamic acid, cysteine, aspartic acid, and homocysteine; and Xaa27 is selected from the group consisting of lysine, leucine, isoleucine, norisoleucine, alanine, methionine, and a polar or hydrophobic amino acid. In a preferred embodiment, Xaa13 is lysine; Xaa17 is glutamic acid; Xaa22 is glutamic acid; Xaa26 is lysine; and/or Xaa27 is leucine. In another preferred embodiment, Xaa22 is glutamic acid (Glu²²), Xaa26 is lysine (Lys²⁶), and Xaa27 is leucine (Leu²⁷).

In another embodiment, the analog consists of the amino acid sequence R-NH-Xaa1-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-Y (SEQ ID NO: 19), wherein R and Y are as described above, and Xaa1 is selected from the group consisting of serine, alanine, and α-aminoisobutyric acid. In a preferred embodiment, Xaa1 is serine.

In another embodiment, the analog consists of the amino acid sequence R-NH-Ser-Val-Ser-Glu-Ile-Gln-Leu-Xaa8-His-Asn-Leu-Gly-Lys-His-Leu-Asn-Ser-Met-Glu-Arg-Val-Glu-Trp-Leu-Arg-Lys-Lys-Leu-Gln-Asp-Val-Y (SEQ ID NO: 20), wherein R and Y are as described above, and Xaa8 is selected from the group consisting of methionine, norisoleucine, and a hydrophobic amino acid. In a preferred embodiment, Xaa8 is methionine.

In another embodiment, the analog consists of the amino acid sequence R-NH-Ser-Val-Ser-Glu-Ile-Gln-Leu-Met-His-Asn-Leu-Gly-Xaa13-His-Leu-Asn-Ser-Met-Glu-Arg-Val-Glu-Trp-Leu-Arg-Lys-Lys-Leu-Gln-Asp-Val-Y (SEQ ID NO: 21), wherein R and Y are as described above, and Xaa13 is selected from the group consisting of lysine, ornithine, glutamic acid, aspartic acid, cysteine, and homocysteine. In a preferred embodiment, Xaa13 is lysine.

In another embodiment, the analog consists of the amino acid sequence R-NH-Ser-Val-Ser-Glu-Ile-Gln-Leu-Met-His-Asn-Leu-Gly-Lys-Xaa14-Leu-Asn-Ser-Met-Glu-Arg-Val-Glu-Trp-Leu-Arg-Lys-Lys-Leu-Gln-Asp-Val-Y (SEQ ID NO: 22), wherein R and Y are as described above, and Xaa14 is histidine or a water soluble amino acid. In a preferred embodiment, Xaa14 is histidine.

In another embodiment, the analog consists of the amino acid sequence R-NH-Ser-Val-Ser-Glu-Ile-Gln-Leu-Met-His-Asn-Leu-Gly-Lys-His-Xaa15-Asn-Ser-Met-Glu-Arg-Val-Glu-Trp-Leu-Arg-Lys-Lys-Leu-Gln-Asp-Val-Y (SEQ ID NO: 23), wherein R and Y are as described above, and Xaa15 is leucine or a water soluble amino acid. In a preferred embodiment, Xaa15 is leucine

In another embodiment, the analog consists of the amino acid sequence R-NH-Ser-Val-Ser-Glu-Ile-Gln-Leu-Met-His-Asn-Leu-Gly-Lys-His-Leu-Xaa16-Ser-Met-Glu-Arg-Val-Glu-Trp-Leu-Arg-Lys-Lys-Leu-Gln-Asp-Val-Y (SEQ ID NO: 24), wherein R and Y are as described above, and Xaa16 is asparagine or a water soluble amino acid. In a preferred embodiment, Xaa16 is asparagine.

In another embodiment, the analog consists of the amino acid sequence R-NH-Ser-Val-Ser-Glu-Ile-Gln-Leu-Met-His-Asn-Leu-Gly-Lys-His-Leu-Asn-Xaa17-Met-Glu-Arg-Val-Glu-Trp-Leu-Arg-Lys-Lys-Leu-Gln-Asp-Val-Y (SEQ ID NO: 25), wherein R and Y are as described above, and Xaa17 is selected from the group consisting of serine, glutamic acid, aspartic acid, lysine, ornithine, cysteine, homocysteine, and a water soluble amino acid. In a preferred embodiment, Xaa17 is glutamic acid or serine.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Xaa18-Glu-Arg-Val-Glu-Trp-Leu-Arg-Lys-Lys-Leu-Gln-Asp-Val-Y (SEQ ID NO: 26), wherein R and Y are as described above, and Xaa18 is selected from the group consisting of methionine, norisoleucine, and a hydrophobic amino acid. In a preferred embodiment, Xaa18 is methionine.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Xaa22-Trp-Leu-Arg-Lys-Lys-Leu-Gln-Asp-Val-Y (SEQ ID NO: 27), wherein R and Y are as described above, and Xaa22 is selected from the group consisting of lysine, ornithine, glutamic acid, cysteine, aspartic acid, and homocysteine. In a preferred embodiment, Xaa22 is glutamic acid.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Xaa25-Lys-Lys-Leu-Gln-Asp-Val-Y (SEQ ID NO: 28), wherein R and Y are as described above, and Xaa25 is arginine or histidine. In a preferred embodiment, Xaa is arginine.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Xaa26-Lys-Leu-Gln-Asp-Val-Y (SEQ ID NO: 29), wherein R and Y are as described above, and Xaa26 is selected from the group consisting of lysine, ornithine, glutamic acid, cysteine, aspartic acid, and homocysteine. In a preferred embodiment, Xaa26 is lysine.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa27-Leu-Gln-Asp-Val-Y (SEQ ID NO: 30), wherein R and Y are as described above, and Xaa27 is selected from the group consisting of lysine, leucine, isoleucine, norisoleucine, alanine, methionine, and a polar or hydrophobic amino acid. In a preferred embodiment, R27 is leucine or lysine.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Xaa22-Trp-Leu-Arg-Xaa26-Lys-Leu-Gln-Asp-Val-Y (SEQ ID NO: 31), wherein R and Y are as described above, and wherein Xaa22 is selected from the group consisting of lysine, ornithine, glutamic acid, cysteine, aspartic acid, and homocysteine and Xaa26 is selected from the group consisting of lysine, ornithine, glutamic acid, cysteine, aspartic acid, and homocysteine. In a preferred embodiment, Xaa22 is glutamic acid and Xaa26 is lysine.

In another embodiment, the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Xaa22-Trp-Leu-Arg-Xaa26-Xaa27-Leu-Gln-Asp-Val-Y (SEQ ID NO: 32), wherein R and Y are as described above, and wherein Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid. In a preferred embodiment, Xaa22 is glutamic acid, Xaa26 is lysine, and Xaa27 is leucine.

In another embodiment, the analog consists of the amino acid sequence R-NH-Ser-Val-Ser-Glu-Ile-Gln-Leu-Met-His-Asn-Leu-Gly-Xaa13-His-Leu-Asn-Xaa17-Met-Glu-Arg-Val-Xaa22-Trp-Leu-Arg-Xaa26-Xaa27-Leu-Gln-Asp-Val-Y (SEQ ID NO: 33), wherein R and Y are as described above, and wherein Xaa13 is selected from the group consisting of lysine, ornithine, glutamic acid, aspartic acid, cysteine, and homocysteine; Xaa17 is selected from the group consisting of serine, glutamic acid, aspartic acid, lysine, ornithine, cysteine, homocysteine, and a water soluble amino acid; Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid. In a preferred embodiment, Xaa13 is lysine, Xaa17 is glutamic acid, Xaa22 is glutamic acid, Xaa26 is lysine, and Xaa27 is leucine.

In another embodiment, the analog consists of the amino acid sequence R-NH-Ser-Val-Ser-Glu-Ile-Gln-Leu-Xaa8-His-Asn-Leu-Gly-Lys-His-Leu-Asn-Ser-Xaa18-Glu-Arg-Val-Xaa22-Trp-Leu-Xaa25-Xaa26-Xaa27-Leu-Gln-Asp-Val-Y (SEQ ID NO: 34), wherein R and Y are as described above, and wherein Xaa8 is selected from the group consisting of methionine, norisoleucine, and a hydrophobic amino acid; Xaa18 is selected from the group consisting of methionine, norisoleucine, and a hydrophobic amino acid; Xaa22 is selected from the group consisting of lysine, ornithine, glutamic acid, cysteine, aspartic acid, and homocysteine; Xaa25 is arginine or histidine; Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid. In a preferred embodiment, Xaa8 is methionine or norisoleucine; Xaa18 is norisoleucine or methionine; and Xaa27 is selected from the group consisting of lysine, leucine, alanine, and norisoleucine.

In another embodiment, the analog consists of the amino acid sequence R-NH-Ser-Val-Ser-Glu-Ile-Gln-Leu-Xaa8-His-Asn-Leu-Gly-Xaa13-His-Leu-Asn-Xaa17-Xaa18-Glu-Arg-Val-Xaa22-Trp-Leu-Xaa25-Xaa26-Xaa27-Leu-Gln-Asp-Val-Y (SEQ ID NO: 35), wherein R and Y are as described above, and wherein Xaa8 is methionine or norisoleucine; Xaa13 is selected from the group consisting of lysine, ornithine, glutamic acid, aspartic acid, cysteine, and homocysteine; Xaa17 is selected from the group consisting of lysine, ornithine, glutamic acid, aspartic acid, cysteine, and homocysteine Xaa18 is methionine or norisoleucine; Xaa22 is selected from the group consisting of lysine, ornithine, glutamic acid, cysteine, aspartic acid, and homocysteine; Xaa25 is arginine or histidine; Xaa26 is selected from the group consisting of lysine, ornithine, glutamic acid, cysteine, aspartic acid, and homocysteine, and Xaa27 is selected from the group consisting of lysine, leucine, norisoleucine, or a polar or hydrophobic amino acid.

In another embodiment, the analog consists of the amino acid sequence R-NH-Ser-Val-Ser-Glu-Ile-Gln-Leu-Met-His-Asn-Leu-Gly-Lys-His-Leu-Asn-Glu-Met-Glu-Arg-Val-Glu-Trp-Leu-Arg-Lys-Xaa27-Leu-Gln-Asp-Val-Y (SEQ ID NO: 36), wherein R and Y are as described above, and wherein Xaa27 is selected from the group consisting of lysine, leucine, isoleucine, norisoleucine, alanine, methionine, and a polar or hydrophobic amino acid. In a preferred embodiment, Xaa27 is leucine.

Preferably, R in any of the above-described analogs is H and X is OH or NH₂. When R is H, the amino terminus is unsubstituted; when X is OH, the C-terminus is a carboxylic acid; and when X is NH₂, the C-terminus is a carboxamide —CONH₂. In another embodiment, any of the above-described analogs are cyclized between the amino acids at positions 13 and 17, 22 and 26, 26 and 30, 27 and 30, or 25 and 29. Examples of hydrophobic amino acids include alanine, valine, phenylalanine, proline, methionine, isoleucine, and leucine; examples of water soluble amino acids include aspartic acid, glutamic acid, lysine, arginine serine, threonine, tyrosine, histidine, cysteine, asparagine, glutamine, and tryptophan; and examples of polar amino acids include serine, threonine, tyrosine, histidine, cysteine, asparagine, glutamine, and tryptophan.

In another embodiment, lysine at amino acid position 27 of SEQ ID NO: 1 is substituted with a polar residue, for example, serine, threonine, tyrosine, histidine, cysteine, asparagine, glutamine, tryptophan, ornithine, or citrulline, a hydrophobic residue, such as alanine, valine, phenylalanine, proline, methionine, leucine, norisoleucine, isoleucine or tyrosine, or a linear or branched α-amino aliphatic acid, having 2-10 carbons in the side chain, or such analogs having a polar or charged group at the terminus of the aliphatic chain. Examples of polar or charged groups include: amino, carboxyl, acetamido, guanido and ureido. Examples of cyclic analogs of hPTH containing amino acid substitutions are described, for example, in U.S. Pat. Nos. 5,955,425, 6,110,892, and 6,316, 410, the entire teachings of which are incorporated herein by reference.

The cyclic hPTH analogs containing such substitutions can be tested for biological activity (e.g., inhibition of skin cell proliferation) as described herein.

Other substitutions of SEQ ID NOs: 1 or 18-36 can be made through conservative amino acid substitutions. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science, 247:1306-1310 (1990). The cyclic hPTH analogs containing such substitutions can be tested for biological activity (e.g., inhibition of skin cell proliferation) as described herein.

A cyclic analog of human parathyroid hormone (hPTH) is also a cyclic peptide of Formula I:
RNH-W-Z-B (I)
where R is hydrogen or a linear or branched chain alkyl, acyl, or aryl group; W is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val ((hPTH-1-31); SEQ ID NO: 1) having 0, 1, 2, 3, 4, 5, 6, 7, 8, 9, or 10 amino acids in the analog differ from the amino acid in the corresponding position of SEQ ID NO:1, Z is selected from the group consisting of His ((hPTH-1-32); SEQ ID NO: 2), His-Asn ((hPTH-1-33); SEQ ID NO: 3), His-Asn-Phe ((hPTH-1-34); SEQ ID NO: 4), His-Asn-Phe-Val ((hPTH-1-35); SEQ ID NO: 5), His-Asn-Phe-Val-Ala ((hPTH-1-36); SEQ ID NO: 6), His-Asn-Phe-Val-Ala-Leu ((hPTH-1-37); SEQ ID NO: 7), His-Asn-Phe-Val-Ala-Leu-Gly ((hPTH-1-38); SEQ ID NO: 8), His-Asn-Phe-Val-Ala-Leu-Gly-Ala ((hPTH-1-39); SEQ ID NO: 9), His-Asn-Phe-Val-Ala-Leu-Gly-Ala-Pro ((hPTH-1-40); SEQ ID NO: 10), His-Asn-Phe-Val-Ala-Leu-Gly-Ala-Pro-Leu ((hPTH-1-41); SEQ ID NO: 11), His-Asn-Phe-Val-Ala-Leu-Gly-Ala-Pro-Leu-Ala ((hPTH-1-42); SEQ ID NO: 12), His-Asn-Phe-Val-Ala-Leu-Gly-Ala-Pro-Leu-Ala-Pro ((hPTH-1-43); SEQ ID NO: 13), and His-Asn-Phe-Val-Ala-Leu-Gly-Ala-Pro-Leu-Ala-Pro-Arg ((hPTH-1-44); SEQ ID NO: 14), and B is OR or NHR. Amino acid substitutions of W are as described herein for hPTH-(1-31) (for example, the substitutions described by SEQ ID NOs: 18-36). In one embodiment, Z has one or more, for example, 2, 3, 4, 5, 6, 7, or 8 amino acid substitutions, for example, conservative amino acid substitutions, as described herein.

The hPTH analog (e.g., peptides represented by SEQ ID NOs: 1, or 18-36) can be cyclized between one or more pairs of amino acids. In one embodiment, the analog is cyclized between one amino acid pair. The amino acid pair can be, for example, the amino acids at positions 22 and 26, 26 and 30, 22 and 25, 22 and 27, 27 and 30, or 25 and 29 of SEQ ID NO: 1. In one embodiment, cyclization occurs through formation of a lactam. In another embodiment, cyclization occurs via disulfide bond formation between the amino acid pair, for example, a pair of cysteine amino acids (e.g., by substituting the amino acids of SEQ ID NO: 1 at the positions where cycliclization will occur with cysteines). In another embodiment, the analog is cyclized between two amino acid pairs. The cyclization can occur, for example, between the amino acids at positions 13 and 17, and 22 and 26 of SEQ ID NO: 1. Again, cyclization can occur through formation of a lactam or through disulfide bond formation between the amino acid pairs, for example, pairs of cysteine amino acids. In preferred embodiments, the cyclic hPTH analog is [Leu²⁷] cyclo(Glu²²-Lys²⁶)-hPTH-(1-31)-NH₂, [Leu²⁷]cyclo(Glu²²-Lys²⁶)-hPTH-(1-32)-NH₂, [Leu²⁷]cyclo(Glu²²-Lys²⁶)-hPTH-(1-33)-NH₂, [Leu²⁷]cyclo(Glu²²-Lys²⁶)-hPTH-(1-34)-NH₂, or [Glu¹⁷, Leu²⁷]cyclo(Lys¹³-Glu¹⁷, Glu²²-Lys²⁶)-hPTH-(1-31)-NH₂, [Glu¹⁷, Leu²⁷]cyclo(Lys¹³-Glu¹⁷, Glu²²-Lys²⁶)-hPTH-(1-32)-NH₂ [Glu¹⁷, Leu²⁷]cyclo(Lys¹³-Glu¹⁷, Glu²²-Lys²⁶)-hPTH-(1-33)-NH₂; or [Glu¹⁷, Leu²⁷]cyclo(Lys¹³-Glu¹⁷, Glu²²-Lys²⁶)-hPTH-(1-34)-NH₂

The present invention also provides methods of treating a condition characterized by hyperproliferation of skin cells by administering a pharmaceutically acceptable salt of a cyclic hPTH analog. Examples of such salts include salts of inorganic acids such as hydrochloric acid and hydrobromic acid, salts of organic acids such as formic acid, acetic acid, tartaric acid and citric acid, salts of inorganic bases such as sodium and ammonium hydroxide and salts of organic bases such as triethylamine, ethylamine, and methylamine.

The cyclic hPTH analogs can be prepared using standard methods for polypeptide production. The cyclic hPTH analogs can be prepared, for example, through synthetic techniques or through recombinant methods. Such methods are described, for example, in U.S. Pat. Nos. 5,955,425, 6,110,892, and 6,316, 410, the entire teachings of which are incorporated herein by reference.

The cyclic analogs of hPTH described herein can be used to treat conditions characterized by hyperproliferation of skin cells. A therapeutically effective amount of the analog can be administered to an individual having the condition to decrease skin cell proliferation. Alternatively, an effective amount of the analog can be prophylactically administered to an individual at risk for having the condition. As used herein, a “therapeutically effective amount” is an amount sufficient to prevent or decrease the proliferation of skin cells, or to improve a condition characterized by hyperproliferation of skin cells. Methods for determining whether a cyclic analog of hPTH is effective (e.g., reducing or eliminating symptoms) in treating a condition characterized by hyperproliferation of skin cells are known to one skilled in the art, and are also described herein.

The terms “therapy,” “therapeutic,” and “treatment” as used herein, refer to ameliorating symptoms associated with a disease or condition, for example, a condition characterized by hyperproliferation of skin cells (e.g., a rate of skin cell proliferation that is 2%, 5%, 10%, 20%, 30%, 40%, 50%, or higher in an individual affected with the condition compared to an unaffected individual), including preventing or delaying the onset of the disease symptoms, and/or lessening the severity or frequency of symptoms of the disease or condition. The terms “subject” and “individual” are defined herein to include animals such as mammals, including, but not limited to, primates, cows, sheep, goats, horses, dogs, cats, rabbits, guinea pigs, rats, mice or other bovine, ovine, equine, canine, feline, rodent, or murine species. In one embodiment, the animal is a human.

In one embodiment, the condition characterized by hyperproliferation of skin cells is psoriasis. The psoriasis can be, for example, erythrodermic psoriasis (also called exfoliative psoriatic dermatitis), or pustular psoriasis. In another embodiment the condition is psoriatic arthritis.

If desired, the cyclic analogs of hPTH can be combined with a pharmaceutically acceptable carrier. Such a carrier is chosen based on the expected route of administration of the composition in therapeutic applications. In one embodiment, the analog is formulated for topical administration. Topical administration includes administration of the analog to the skin at the site of the condition. For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The cyclic analog of hPTH can also be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

In another embodiment, administration of the cyclic analog of hPTH is oral, lingual, sublingual, buccal or intrabuccal. Such administration can be made without undue experimentation by means well known in the art, for example, with an inert diluent or with an edible carrier. The cyclic analog can be enclosed in gelatin capsules or compressed into tablets. For the purpose of oral therapeutic administration, cyclic analogs can be incorporated with excipients and used in the form of tablets, troches, capsules, elixirs, suspensions, syrups, wafers, chewing gums and the like. The tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used.

The combination therapy compositions of the present invention can be administered parenterally such as, for example, by intravenous, intramuscular, intrathecal, or subcutaneous injection. Parenteral administration can be accomplished by incorporating the cyclic analogs into a solution or suspension. Such solutions or suspensions may also include sterile diluents such as water for injection, saline solution, fixed oils, polyethylene glycols, glycerine, propylene glycol, or other synthetic solvents. Buffers such as acetates, citrates, or phosphates and agents for the adjustment of tonicity such as sodium chloride or dextrose may also be added.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the cyclic analogs of hPTH.

The dosage of the combination therapy compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Typically, a therapeutically effective amount can range from 0.01 mg per day to about 100 mg per day for an adult. Preferably, the dosage ranges from about 1 mg per day to about 100 mg per day or from about 1 mg per day to about 10 mg per day.

If desired, the analog can be combined or coadministered with one or more additional agents used to treat a condition characterized by hyperproliferation of skin cells. Such agents are know to one of skill in the art. The agent can be, for example, alclometasone depropionate, hydrocortisone, calcipotriene, cyclosporine, methotrexate, methoxsalen, anthralin, acitretin, tazarotene, or clobetasol propionate. The analog can be formulated in the same preparation as the additional agent or the analog and the additional agent(s) can be formulated separately and administer to the individual simultaneously or consecutively, in either order.

Exemplification

The present invention will now be illustrated by the following Examples, which are not intended to be limiting in any way.

### EXAMPLE 1

### Topical Administration of the Human Parathyroid Hormone hPTH Analog cyclo(Lys²⁶-Asp³⁰) [Leu²⁷]-hPTH-(1-31)-NH₂ Decreases Skin Cell Proliferation

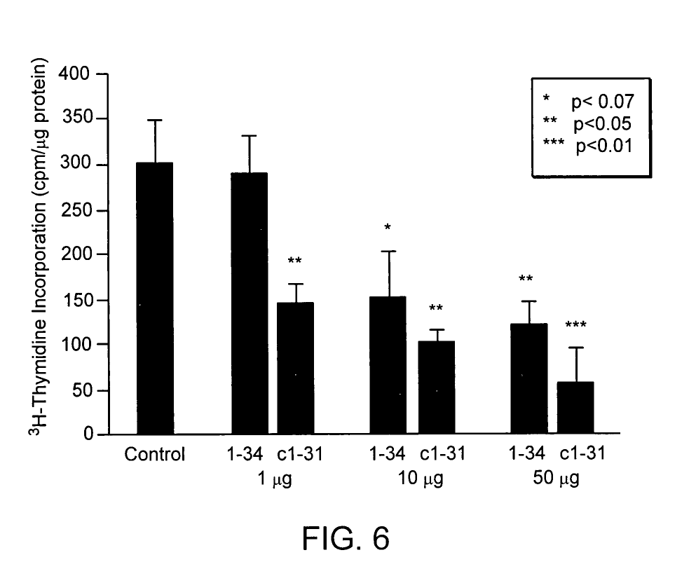

SKH-1 hairless mice were administered the human parathyroid hormone hPTH analog [Leu²⁷]cyclo(Glu²²-Lys²⁶)-hPTH-(1-31)-NH₂ with Novasome (a liposomal formulation available, for example, from IGI, Inc., Buena, N.J.) or hPTH-(1-34) at dosages of 1 μg, 10 μg, or 50 μg once a day, or were left untreated (control). On day 7, the mice were injected twice with ³H-thymidine, and on day 8, the mice were injected with ³H-thymidine and bromodeoxyuridine (1.5 mg/mouse) and sacrificed within 3 hours. ³H-thymidine incorporation was then assessed, using standard methods. The results of this study are shown in FIG. 6, which is a histogram showing the effects of no treatment (Control) or of topical administration of 1 μg, 10 μg, or 50 μg of hPTH-(1-34) (1-34) or [Leu²⁷]cyclo(Glu²²-Lys²⁶)-hPTH-(1-31)-NH₂ (c1-c31) on proliferation of skin cells, as measured by ³H-thymidine incorporation (counts per minute/μg protein) in SKH-1 mice. As shown in FIG. 6, [Leu²⁷]cyclo(Glu²²-Lys²⁶)-hPTH-(1-31)-NH₂ was significantly more effective at inhibiting ³H-thymidine incorporation than hPTH-(1-34). These results demonstrate that cyclic analogs of hPTH can be used to decrease skin cell proliferation. As such, these cyclic analogs can be used to treat conditions characterized by hyperproliferation of skin cells.

While this invention has been particularly shown and described with references to preferred embodiments thereof, it will be understood by those skilled in the art that various changes in form and details may be made therein without departing from the scope of the invention encompassed by the appended claims.

1. A method of treating a condition characterized by hyperproliferation of skin cells in an individual at risk for or having the condition comprising administering a therapeutically effective amount of a cyclic analog of human parathyroid hormone (hPTH) consisting of the amino acid sequence: R-NH-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-Y (SEQ ID NO: 1), wherein R is hydrogen or a linear or branched chain alkyl, acyl, or aryl group; and Y is X, His-X, His-Asn-X, or His-Asn-Phe-X, wherein X is OR or NHR, and having 0, 1, 2, 3, 4, 5, 6, 7, 8, 9, 10, 11, or 12 amino acids in the analog differ from the amino acid in the corresponding position of SEQ ID NO: 1 or a pharmaceutically acceptable salt thereof to the individual.

2. The method of claim 1, wherein the analog or pharmaceutically acceptable salt thereof consists of the sequence of SEQ ID NO: 1.

3. The method of claim 1, wherein the analog consists of the amino acid sequence R-NH-Xaa1-Val-Ser-Glu-Ile-Gln-Leu-Xaa8-His-Asn-Leu-Gly-Xaa13-Xaa14-Xaa15-Xaa16-Xaa17-Xaa18-Glu-Arg-Val-Xaa22-Trp-Leu-Xaa25-Xaa26-Xaa27-Leu-Gln-Asp-Val-Y (SEQ ID NO: 18), wherein Xaa1 is selected from the group consisting of serine, alanine, and α-aminoisobutyric acid; Xaa8 is selected from the group consisting of methionine, norisoleucine, and a hydrophobic amino acid; Xaa13 is selected from the group consisting of lysine, ornithine, glutamic acid, aspartic acid, cysteine, and homocysteine; Xaa14 is histidine or a water soluble amino acid; Xaa15 is leucine or a water soluble amino acid; Xaa16 is asparagine or a water soluble amino acid; Xaa17 is selected from the group consisting of serine, glutamic acid, aspartic acid, lysine, ornithine, cysteine, homocysteine, and a water soluble amino acid; Xaa18 is selected from the group consisting of methionine, norisoleucine, and a hydrophobic amino acid; Xaa22 is selected from the group consisting of lysine, ornithine, glutamic acid, cysteine, aspartic acid, and homocysteine; Xaa25 is arginine or histidine; Xaa26 is selected from the group consisting of lysine, ornithine, glutamic acid, cysteine, aspartic acid, and homocysteine; and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

4. The method of claim 3, wherein the analog or pharmaceutically acceptable salt thereof is cyclized between one amino acid pair.

5. The method of claim 4, wherein the amino acid pair is selected from the group consisting of the amino acids at Xaa22 and Xaa26, Xaa26 and Xaa30, Xaa27 and Xaa30, and Xaa25 and Xaa29 of SEQ ID NO: 18.

6. The method of claim 5, wherein the amino acid pair consists of the amino acids at Xaa22 and Xaa26 of SEQ ID NO: 18.

7. The method of claim 6, wherein the analog consists of the amino acid sequence R-NH-Ser-Val-Ser-Glu-Ile-Gln-Leu-Met-His-Asn-Leu-Gly-Xaa13-His-Leu-Asn-Xaa17-Met-Glu-Arg-Val-Xaa22-Trp-Leu-Arg-Xaa26-Xaa27-Leu-Gln-Asp-Val-Y (SEQ ID NO: 33), wherein Xaa13 is selected from the group consisting of lysine, ornithine, glutamic acid, aspartic acid, cysteine, and homocysteine; Xaa17 is selected from the group consisting of serine, glutamic acid, aspartic acid, lysine, ornithine, cysteine, homocysteine, and a water soluble amino acid; Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

8. The method of claim 7, wherein Xaa22 of SEQ ID NO: 33 and Xaa26 of SEQ ID NO: 33 are cysteines, and wherein the analog has been cyclized between the thiol groups of the cysteines to form a disulfide bond.

9. The method of claim 6, wherein the analog has been cyclized between the amino acids at Xaa22 and Xaa26 to form a lactam.

10. The method of claim 9, wherein the analog consists of the amino acid sequence R-NH-Ser-Val-Ser-Glu-Ile-Gln-Leu-Xaa8-His-Asn-Leu-Gly-Lys-His-Leu-Asn-Ser-Xaa18-Glu-Arg-Val-Xaa22-Trp-Leu-Xaa25-Xaa26-Xaa27-Leu-Gln-Asp-Val-Y (SEQ ID NO: 34), wherein Xaa8 is selected from the group consisting of methionine, norisoleucine, and a hydrophobic amino acid; Xaa18 is selected from the group consisting of methionine, norisoleucine, and a hydrophobic amino acid; Xaa22 is selected from the group consisting of lysine, ornithine, glutamic acid, cysteine, aspartic acid, and homocysteine; Xaa25 is arginine or histidine; Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

11. The method of claim 9, wherein the analog consists of the amino acid sequence R-NH-Ser-Val-Ser-Glu-Ile-Gln-Leu-Met-His-Asn-Leu-Gly-Xaa13-His-Leu-Asn-Xaa17-Met-Glu-Arg-Val-Xaa22-Trp-Leu-Arg-Xaa26-Xaa27-Leu-Gln-Asp-Val-Y (SEQ ID NO: 33), wherein Xaa13 is selected from the group consisting of lysine, ornithine, glutamic acid, aspartic acid, cysteine, and homocysteine; Xaa17 is selected from the group consisting of serine, glutamic acid, aspartic acid, lysine, ornithine, cysteine, homocysteine, and a water soluble amino acid; Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

12. The method of claim 11, wherein Xaa13 is lysine, Xaa17 is glutamic acid, Xaa22 is glutamic acid, Xaa26 is lysine, and Xaa27 is leucine.

13. The method of claim 9, wherein the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Xaa22-Trp-Leu-Arg-Xaa26-Xaa27-Leu-Gln-Asp-Val-Y (SEQ ID NO: 32) wherein Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

14. The method of claim 9, wherein the analog consists of the amino acid sequence R-NH-Ser-Val-Ser-Glu-Ile-Gln-Leu-Met-His-Asn-Leu-Gly-Lys-His-Leu-Asn-Ser-Met-Glu-Arg-Val-Xaa22-Trp-Leu-Arg-Xaa26-Lys-Leu-Gln-Asp-Val-Y (SEQ ID NO: 31), wherein Xaa22 is selected from the group consisting of lysine, ornithine, glutamic acid, cysteine, aspartic acid, and homocysteine and Xaa26 is selected from the group consisting of lysine, ornithine, glutamic acid, cysteine, aspartic acid, and homocysteine, and wherein R is hydrogen or a linear or branched chain alkyl, acyl, or aryl group; and Y is X, His-X, His-Asn-X, or His-Asn-Phe-X, wherein X is OR or NHR.

15. The method of claim 9, wherein R is H.

16. The method of claim 9, wherein Y is X.

17. The method of claim 16, wherein X is NH₂.

18. The method of claim 17, wherein R is H.

19. The method of claim 3, wherein the analog or pharmaceutically acceptable salt thereof is cyclized between two amino acid pairs.

20. The method of claim 19, wherein the amino acid pairs are the amino acids at Xaa13 and Xaa17, and Xaa22 and Xaa26 of SEQ ID NO: 18.

21. The method of claim 20, wherein the analog consists of the amino acid sequence R-NH-Ser-Val-Ser-Glu-Ile-Gln-Leu-Met-His-Asn-Leu-Gly-Xaa13-His-Leu-Asn-Xaa17-Met-Glu-Arg-Val-Xaa22-Trp-Leu-Arg-Xaa26-Xaa27-Leu-Gln-Asp-Val-Y (SEQ ID NO: 33), wherein Xaa13 is selected from the group consisting of lysine, ornithine, glutamic acid, aspartic acid, cysteine, and homocysteine; Xaa17 is selected from the group consisting of serine, glutamic acid, aspartic acid, lysine, ornithine, cysteine, homocysteine, and a water soluble amino acid; Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

22. The method of claim 21, wherein Xaa22 of SEQ ID NO: 33 and Xaa26 of SEQ ID NO: 33 are cysteines, and wherein the analog has been cyclized between the thiol groups of the cysteines to form a disulfide bond.

23. The method of claim 21, wherein Xaa13 of SEQ ID NO: 33 and Xaa17 of SEQ ID NO: 33 are cysteines, and wherein the analog has been cyclized between the thiol groups of the cysteines to form a disulfide bond.

24. The method of claim 21, wherein Xaa13 of SEQ ID NO: 33, Xaa17 of SEQ ID NO: 33, Xaa22 of SEQ ID NO: 33 and Xaa26 of SEQ ID NO: 33 are cysteines, and wherein the analog has been cyclized between the thiol groups of the cysteines of Xaa13 and Xaa17, and of Xaa22 and Xaa26 to form disulfide bonds.

25. The method of claim 20, wherein the analog has been cyclized between the amino acids at positions Xaa13 and Xaa17, and Xaa22 and Xaa26 to form a lactam.

26. The method of claim 25, wherein Xaa17 is glutamic acid.

27. The method of claim 25, wherein the analog consists of the amino acid sequence R-NH-Ser-Val-Ser-Glu-Ile-Gln-Leu-Met-His-Asn-Leu-Gly-Lys-His-Leu-Asn-Glu-Met-Glu-Arg-Val-Glu-Trp-Leu-Arg-Lys-Xaa27-Leu-Gln-Asp-Val-Y (SEQ ID NO: 36), wherein Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

28. The method of claim 25, wherein the analog consists of the amino acid sequence R-NH-Ser-Val-Ser-Glu-Ile-Gln-Leu-Met-His-Asn-Leu-Gly-Xaa13-His-Leu-Asn-Xaa17-Met-Glu-Arg-Val-Xaa22-Trp-Leu-Arg-Xaa26-Xaa27-Leu-Gln-Asp-Val-Y (SEQ ID NO: 33), wherein Xaa13 is selected from the group consisting of lysine, ornithine, glutamic acid, aspartic acid, cysteine, and homocysteine; Xaa17 is selected from the group consisting of serine, glutamic acid, aspartic acid, lysine, ornithine, cysteine, homocysteine, and a water soluble amino acid; Xaa22 is selected from the group consisting of lysine, ornithine, glutamic acid, cysteine, aspartic acid, and homocysteine, Xaa26 is selected from the group consisting of lysine, ornithine, glutamic acid, cysteine, aspartic acid, and homocysteine, and Xaa27 is selected from the group consisting of lysine, leucine, isoleucine, norisoleucine, alanine, methionine, and a polar or hydrophobic amino acid, and wherein R is hydrogen or a linear or branched chain alkyl, acyl, or aryl group; and Y is X, His-X, His-Asn-X, or His-Asn-Phe-X, wherein X is OR or NHR.

29. The method of claim 28, wherein Xaa13 is lysine, Xaa17 is glutamic acid, Xaa22 is glutamic acid, Xaa26 is lysine, and Xaa27 is leucine.

30. The method of claim 25, wherein R is H.

31. The method of claim 25, wherein Y is X.

32. The method of claim 31, wherein X is NH₂.

33. The method of claim 32, wherein R is H.

34. The method of claim 1, wherein the condition characterized by hyperproliferation of the skin is selected from the group consisting of psoriasis, psoriatic arthritis, and erythrokeratodermia variabilis.

35. The method of claim 34, wherein the condition characterized by hyperproliferation of the skin is psoriasis.

36. The method of claim 1, wherein the analog or pharmaceutically acceptable salt thereof is in a pharmaceutically acceptable carrier.

37. A method of treating a condition characterized by hyperproliferation of skin cells in an individual at risk for or having the condition comprising administering a therapeutically effective amount of the human parathyroid hormone hPTH analog cyclo(Glu²²-Lys²⁶)[Leu²⁷]-hPTH-(1-31)-NH₂ or a pharmaceutically acceptable salt thereof to the individual.

38. The method of claim 37, wherein the condition characterized by hyperproliferation of the skin is psoriasis.

39. The method of claim 37, wherein the analog or pharmaceutically acceptable salt thereof is in a pharmaceutically acceptable carrier.

40. A method of treating a condition characterized by hyperproliferation of skin cells in an individual at risk for or having the condition comprising administering a therapeutically effective amount of a cyclic analog of human parathyroid hormone (hPTH) of Formula I:
RNH-W-Z-B (I)
wherein R is hydrogen or a linear or branched chain alkyl, acyl, or aryl group; W is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val (SEQ ID NO: 1) having 0, 1, 2, 3, 4, 5, 6, 7, 8, 9, 10, 11, or 12 amino acids in the analog differ from the amino acid in the corresponding position of SEQ ID NO: 1, Z is selected from the group consisting of His, His-Asn, His-Asn-Phe, His-Asn-Phe-Val (SEQ ID NO: 5), His-Asn-Phe-Val-Ala (SEQ ID NO: 6), His-Asn-Phe-Val-Ala-Leu (SEQ ID NO: 7), His-Asn-Phe-Val-Ala-Leu-Gly (SEQ ID NO: 8), His-Asn-Phe-Val-Ala-Leu-Gly-Ala (SEQ ID NO: 9), His-Asn-Phe-Val-Ala-Leu-Gly-Ala-Pro (SEQ ID NO: 10), His-Asn-Phe-Val-Ala-Leu-Gly-Ala-Pro-Leu (SEQ ID NO: 11), His-Asn-Phe-Val-Ala-Leu-Gly-Ala-Pro-Leu-Ala (SEQ ID NO: 12), His-Asn-Phe-Val-Ala-Leu-Gly-Ala-Pro-Leu-Ala-Pro (SEQ ID NO: 13), and His-Asn-Phe-Val-Ala-Leu-Gly-Ala-Pro-Leu-Ala-Pro-Arg (SEQ ID NO: 14), and B is OR or NHR, or a pharmaceutically acceptable salt thereof to the individual.
